(19) United States
(12) Patent Application Publication
(10) Pub. No.: US 2011/0240017 A1
(43) Pub. Date: Oct. 6, 2011

(54) APPARATUS AND METHODS FOR MICROBARIC OXYGEN DELIVERY

(76) Inventors: Glenn Butler, Tarrytown, NY (US); Russell E. Peterson, Alpharetta, GA (US); Michael William Allen, Furlong, PA (US); Jacques Vieweg, Tarrytown, NY (US)

(21) Appl. No.: 13/078,776

(22) Filed: Apr. 1, 2011

Related U.S. Application Data
(60) Provisional application No. 61/320,570, filed on Apr. 2, 2010.

(51) Int. Cl. A61G 10/00; A61M 16/00

(52) U.S. Cl. 128/201.25; 128/201.23

(57) ABSTRACT

A system is provided for providing oxygen from an oxygen supply to a user. The system includes: (a) a hood adapted to fit over the user's head. The hood includes an inlet port, an outlet port, and a seal mechanism adapted to engage at least one of the user's head, neck and torso to restrict a flow of oxygen from the hood; (b) a flow control mechanism for coupling between the oxygen supply and the inlet port, and for controlling a flow of oxygen to the hood; and (c) a pressure control mechanism coupled to the outlet port, wherein the back pressure valve is adapted to regulate a pressure of the oxygen in the hood to between about 0.5 inches of water (“inH₂O”) (0.00127 atm) and about 4 inH₂O (0.01016 atm). Numerous other aspects are provided.

### REFERENCE TO RELATED APPLICATIONS

This application claims the benefit of U.S. Provisional Patent Application Ser. No. 61/320,570, filed 2 Apr. 2010, which is incorporated by reference herein in its entirety for all purposes.

### BACKGROUND

This invention relates to apparatus and methods for microbaric oxygen delivery. More particularly, this invention relates to apparatus and methods for microbaric oxygen delivery for treatment of acute and/or chronic neurological conditions.

Research has shown that hyperbaric oxygen therapy can benefit a number of neurologic conditions, including cerebral palsy, autism, traumatic brain injury, stroke, spinal cord injury, chronic fatigue syndrome, fibromyalgia, reflex sympathetic dystrophy, migraine and cluster headaches, multiple sclerosis, and certain types of dementia.

Conventional hyperbaric oxygen delivery systems, such as hyperbaric oxygen chambers, however, have several drawbacks. In particular, conventional hyperbaric oxygen delivery systems typically are quite large, bulky and expensive, and may subject the patient to risks associated with full-body exposure to increased ambient pressures. Thus, improved apparatus and methods for therapeutic administration of increased partial pressures of oxygen are desirable.

### SUMMARY

A first aspect of the invention provides a system for delivering oxygen from an oxygen supply to a user. The system includes: (a) a hood adapted to fit over the user's head. The hood includes an inlet port, an outlet port, and a seal mechanism adapted to engage at least one of the user's head, neck and torso to restrict a flow of oxygen from the hood; (b) a flow control mechanism for coupling between the oxygen supply and the inlet port, and for controlling a flow of oxygen to the hood; and (c) a pressure control mechanism coupled to the outlet port, wherein the pressure control mechanism is adapted to regulate a pressure of the oxygen in the hood to between about 0.5 inches of water (“inH₂O”) (0.00127 atm) and about 4 inH₂O (0.01016 atm).

In a second aspect of the invention, a method is provided for delivering oxygen from an oxygen supply to a user. The method includes: (a) providing a hood adapted to fit over the user's head, the hood comprising an inlet port, an outlet port, and a seal mechanism adapted to engage at least one of the user's head, neck and torso to restrict a flow of oxygen from the hood; (b) controlling a flow of oxygen to the inlet port of the hood; and (c) regulating a pressure of the oxygen in the hood from the outlet port to between about 0.5 inH₂O (0.00127 atm) and about 4 inH₂O (0.01016 atm).

In a third aspect of the invention, a system is provided for use with a counterlung for delivering oxygen from an oxygen supply to a user. The counter lung has a first inlet port, a second inlet port, and an outlet port. The system includes: (a) a hood adapted to fit over the user's head, the hood comprising an inlet port, an outlet port, and a seal mechanism adapted to engage at least one of the user's head, neck and torso to restrict a flow of oxygen from the hood; (b) a flow control mechanism adapted to couple between the oxygen supply and the first inlet port of the counter lung, and to control a flow of oxygen to the hood; (c) a first pressure control mechanism coupled to the outlet port of the hood, wherein the first pressure control mechanism is adapted to regulate a pressure of the oxygen in the hood to between about 0.5 inH₂O (0.00127 atm) and about 4 inH₂O (0.01016 atm); and (d) a carbon dioxide scrubber adapted to couple between the second inlet port of the counter lung and an outlet port of the first pressure control mechanism.

Other features and aspects of the invention will become more fully apparent from the following detailed description, the appended claims, and the accompanying drawings.

### BRIEF DESCRIPTION OF THE DRAWINGS

Features of the present invention can be more clearly understood from the following detailed description considered in conjunction with the following drawings, in which the same reference numerals denote the same elements throughout, and in which:

FIG. 1 is diagram of an example of a microbaric oxygen delivery system in accordance with this invention;

FIG. 2 is a diagram of an example embodiment of the control system of FIG. 1;

FIG. 3 is a diagram of an alternative example embodiment of the control system of FIG. 1; and

FIG. 4 is a diagram of an example equipment housing of the control system of FIG. 1.

### DETAILED DESCRIPTION

This invention provides apparatus and methods for microbaric oxygen delivery for treating neurological conditions. Example apparatus include a hood, a flow control mechanism, and a pressure control mechanism. The hood is adapted to fit over a user's head, and includes an inlet port, an outlet port, and a seal mechanism adapted to engage the user's head, neck and/or torso to restrict a flow of oxygen from the hood. The flow control mechanism is adapted to couple to an oxygen supply and the inlet port of the hood, and controls a flow of oxygen to the hood. The pressure control mechanism is coupled to the outlet port of the hood, and regulates a pressure of the oxygen in the hood to a pressure between about 0.5 inH₂O (0.00127 atm) and about 4 inH₂O (0.01016 atm). Example apparatus may be used to provide microbaric oxygen delivery to patients for treatment of neurological conditions.

Research has shown that therapeutic administration of increased partial pressures of oxygen can benefit a number of neurologic conditions, including cerebral palsy, autism, traumatic brain injury, stroke, spinal cord injury, chronic fatigue syndrome, fibromyalgia, reflex sympathetic dystrophy, migraine and cluster headaches, multiple sclerosis, and certain types of dementia.

In some cases, the oxygen levels used to produce such positive impact have been slightly higher than that provided by air at one atmosphere. This has been most evident in studies of the effects of hyperoxia on cerebral palsy and autism. This latter research has been conducted with increased ambient pressures, with a focus on administering oxygen doses that only may be achieved under hyperbaric conditions. See Daniel A. Rossignol et al. “Hyperbaric Treatment For Children With Autism: A Multicenter, Randomized, Double-Blind, Controlled Trial,” BMC Pediatrics 2009, 9:21 (Mar. 13, 2009) and Dr. Pierre Marois et al. “Hyperbaric Oxygen Therapy and Cerebral Palsy,” Developmental Medicine & Child Neurology, 45:646-648 (2003).

In the course of these studies, however, it has been discovered that administration to “control” subjects of lower oxygen levels (e.g., ones achievable with slightly higher than ambient pressure conditions), produces outcomes statistically indistinguishable from those achieved with the higher, experimental oxygen levels. Further, the outcomes from both test conditions have been significantly better than therapy programs without any hyperoxic treatment.

Because there is no effect of increased ambient pressure or other therapy aspect that could mimic the impact of hyperoxia in neurological cases such as cerebral palsy and autism, it is believed that treatment with pure oxygen at low hyperbaric pressure (referred to herein as “microbaric”) offers the potential for great benefit to such patients. As used herein, “microbaric” pressure means between about 0.5 inH₂O (0.00127 atm) and about 4 inH₂O (0.01016 atm).

Conventional hyperbaric oxygen therapies usually are administered using a hyperbaric chamber, which is a transparent, cylindrical chamber, approximately 8 feet long and 3 feet in diameter. The patient lays on a cot-like stretcher, and is then rolled into the chamber. During treatment, the patient is surrounded by and inhales pure oxygen, while pressure within the chamber is increased from 1½ to 2 times the outside pressure. At the end of treatment, the patient is gradually decompressed to normal pressure, and is then removed from the chamber.

Although conventional hyperbaric chambers may be used for microbaric oxygen therapies, such chambers have several disadvantages that limit their effectiveness for microbaric oxygen therapy. Conventional hyperbaric chambers are large, expensive, and are typically found only in hospitals and specialized medical offices. Thus, hyperbaric chambers may not easily or cost-effectively be used in other environments, such as at home, or outdoors (e.g., in battle, or at the scene of an accident). Further, the patient typically must incur the cost and inconvenience of travelling to the site of the hyperbaric chamber. Additionally, although conventional hyperbaric chambers are generally safe, full-body exposure to hyperbaric pressures may cause the patient to experience confinement anxiety, claustrophobia, hyperthermia, hypothermia, or other problems.

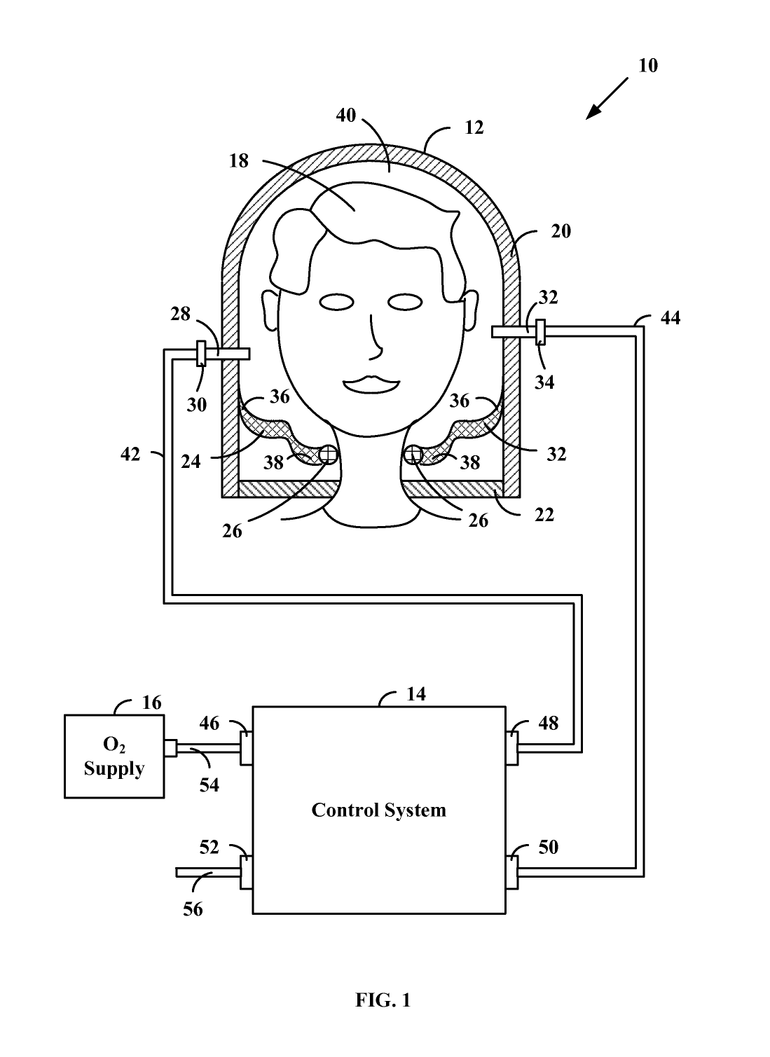

Apparatus and methods in accordance with this invention do not have these disadvantages. Referring now to FIG. 1, an example of a microbaric oxygen delivery system 10 in accordance with this invention is described. Microbaric oxygen delivery system 10 includes a hood 12 and a control system 14, which is coupled to a supply 16 of substantially pure oxygen, such as United States Pharmacopoeia (“USP”) medical grade oxygen.

Hood 12 is adapted to fit over the head of a user 18, and includes a dome portion 20 having a bottom opening 22, a flexible baffle 24 having a seal mechanism 26, an inlet port 28 having a coupling mechanism 30, an outlet port 32 having a coupling mechanism 34.

Dome portion 20 may be a clear lightweight material that may be used to restrict flow of a fluid, such as oxygen. For example, dome portion 20 may be glass, plastic, vinyl, Plexiglas or other similar material. Bottom opening 22 may have a circular, elliptical, rectangular or other similar shape opening, and should be sized to fit over and/or around the head of user 18. Persons of ordinary skill in the art will understand that all or any of dome portion 20 need not be clear. For example, dome portion 20 may be metal or other similar material. Additionally, although depicted in FIG. 1 as having a bell-jar shape, dome portion 20 may have other shapes.

Flexible baffle 24 may have a torroidal or other similar shape, with a first end 36 attached to an interior sidewall of dome portion 20, and a second end 38 attached to seal mechanism 26. Flexible baffle 24 may be a lightweight flexible material that may be used to restrict flow of a fluid, such as oxygen. For example, flexible baffle 24 may be a plastic, vinyl or other similar material. Flexible baffle 24 may have a shape other than torroidal.

Seal mechanism 26 may be a ring-shaped or otherwise suitably shaped flexible material with memory having an expandable opening that may be stretched to fit over the head of user 18, but that contracts to engage the head, neck and/or torso of user 18 to restrict flow of fluid (e.g., e.g., oxygen or other similar fluid) from hood 12. For example, seal mechanism 26 may be a plastic, Teflon, rubber or other similar material.

Hood 12 is configured so that user 18 inserts her head through bottom opening 22 and through the expandable opening of seal mechanism 26, which contracts to engage the head, neck and/or torso of user 18 to form a substantially sealed inner chamber 40 about the head of user 18. Although not shown in FIG. 1, bottom opening may have one or more straps, harnesses, or other mechanisms to secure hood 12 to user 18 to prevent hood 12 from falling or becoming dislodged from user 18.

Inlet port 28 and outlet port 32 may be disposed on sidewall portions of dome portion 20, and may be hollow cylindrical openings for allowing a fluid (e.g., oxygen, patient exhalation, or other similar fluid) to flow in and out of chamber 40. Persons of ordinary skill in the art will understand that inlet port 28 and outlet port 32 may have shapes other than cylindrical.

Coupling mechanisms 30 and 34 may be used to attach hoses 42 and 44, respectively, to inlet port 28 and outlet port 32, respectively. Coupling mechanisms 30 and 34 may include quick-connect or other similar mechanisms that permit easy connection to and disconnection from hoses 42 and 44, respectively.

Although not shown in FIG. 1, any of inlet port 28, outlet port 32, and coupling mechanisms 30 and 34 may include a check valve for restricting the direction of flow of fluid (e.g., oxygen, carbon dioxide, or other similar fluid) into or out of hood 12. In addition, although not shown in FIG. 1, any of inlet port 28, outlet port 32, and coupling mechanisms 30 and 34 may include a filter for filtering fluid (e.g., oxygen, carbon dioxide, or other similar fluid) flowing in or out of hood 12.

Control system 14 includes a first inlet port 46, a first outlet port 48, a second inlet port 50 and a second outlet port 52. First inlet port 46 may be coupled via a hose 54 to oxygen supply 16, first outlet port 48 may be coupled via hose 42 and coupling mechanism 30 to inlet port 28, second inlet port 50 may be coupled via hose 44 and coupling mechanism 34 to outlet port 32, and second outlet port 52 may be coupled via hose 56 to atmosphere. As described in more detail below, control system 14 controls a flow of oxygen from oxygen supply 16 to hood 12, and regulates a pressure of oxygen in hood 12 to between about 0.5 inH₂O (0.00127 atm) and about 4 inH₂O (0.01016 atm).

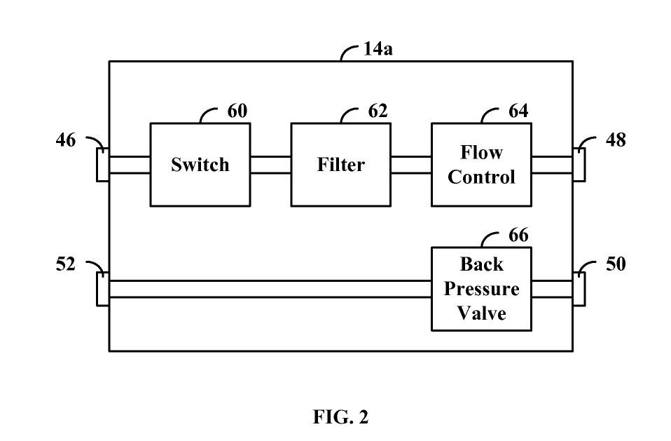

Referring now to FIG. 2, an example control system 14a is described. Control system 14a includes a switch 60, a filter 62 and a flow control mechanism 64 coupled in series between first inlet port 46 and first outlet port 48, and a pressure control mechanism 66 coupled between second inlet port 50 and second outlet port 52.

Switch 60 may be a pneumatic or other similar switch that may be used to turn ON or OFF the flow of oxygen from oxygen supply 16 (FIG. 1) to filter 62 and flow control mechanism 64. For example, switch 60 may be a model MTV-2P pneumatic switch manufactured by Clippard Instrument Laboratory, Inc., Cincinnati, Ohio, although other pneumatic switches may be used.

Filter 62 may be used to filter any particulate or other unwanted matter in the flow of oxygen from oxygen supply 16. For example, filter 62 may be a model PN154 filter manufactured by Sea-Long Medical Systems, Inc., Louisville, Ky., although other filters may be used. Persons of ordinary skill in the art will understand that filter 62 optionally may be omitted.

Flow control mechanism 64 may be used to control a flow of oxygen from oxygen supply 16 to hood 12. For example, flow control mechanism 64 may be a model MMA-25 flow control mechanism manufactured by Dwyer Instruments, Inc., Michigan City, Ind., although other flow control mechanisms may be used.

Pressure control mechanism 66 may be used to regulate a pressure of oxygen in hood 12 to provide microbaric oxygen delivery to user 18. In particular, pressure control mechanism 66 may include a control mechanism (not shown in FIG. 2) for setting a desired oxygen pressure level between about 0.5 inH₂O (0.00127 atm) and about 4 inH₂O (0.01016 atm) in chamber 40.

Pressure control mechanism 66 may be a back pressure valve, a relief valve, backpressure regulator, or other similar device to regulate upstream (back) pressure. For example, pressure control mechanism 66 may be a model 9005B ACCU-PEEP™ back pressure valve manufactured by Vital Signs, Inc, Totowa, N.J., although other pressure control mechanisms may be used.

Referring now to FIGS. 1 and 2, switch 60 may be used turn ON the flow of oxygen from oxygen supply 16. Filter 62 removes particulate or other unwanted matter in the flow of oxygen, which is regulated by flow control mechanism 64 to provide a desired flow rate to hood 12 via first outlet port 48 and hose 42. Oxygen begins filling chamber 40, and user 18 inhales oxygen from chamber 40, and exhales fluid (e.g., carbon dioxide) into chamber 40. The exhaled fluid and residual oxygen in chamber 40 returns via tube 44 and second inlet port 50 to pressure control mechanism 66, which regulates pressure inside chamber 40 to a desired microbaric pressure level. Any excess flow of return fluid is exhausted to atmosphere via second outlet port 52 and tube 56.

The return fluid flow from hood 12 includes a mixture of oxygen and carbon monoxide. Thus, some portion of the return fluid that is exhausted by pressure control mechanism 66 to atmosphere includes oxygen. In this regard, example control system 14a may be described as an “open” system. To avoid such waste, an alternative example “closed” control system 14 may be used to re-circulate the exhausted oxygen back into control system 14 for delivery to hood 12.

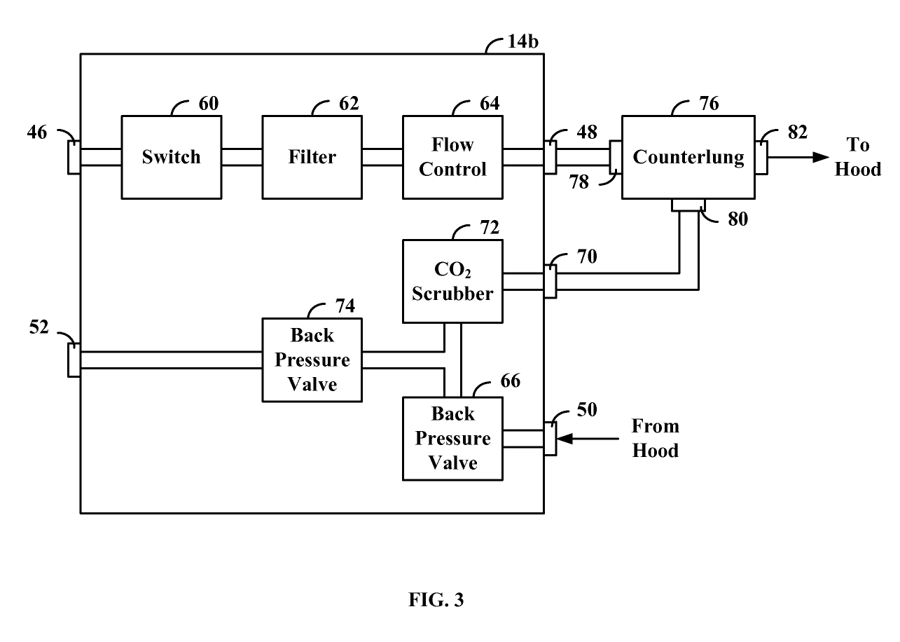

Referring now to FIG. 3, an example “closed” control system 14b is described. Control system 14b is similar to control system 14a, but also includes a third outlet port 70, a carbon dioxide (CO₂) scrubber 72 and a second pressure control mechanism 74. Pressure control mechanism 66 has an outlet coupled to an inlet of CO₂ scrubber 72 and an inlet of pressure control mechanism 74. CO₂ scrubber 72 has an outlet coupled to third outlet port 70.

CO₂ scrubber 72 may be used to receive at its inlet a mixture of oxygen and carbon dioxide, and substantially removes carbon dioxide from the mixture, to provide oxygen at its outlet port. For example, CO₂ scrubber 72 may be a fabricated using Drägersorb 800+ soda lime canisters manufactured by Draeger Medical Inc., Telford, Pa., although other soda lime canisters may be used.

Pressure control mechanism 74 may be used to regulate a pressure of the closed system. In particular, pressure control mechanism 74 may include a control mechanism (not shown in FIG. 3) for setting a desired system pressure level to between 0.5 inH₂O (0.00127 atm) and about 4.5 inH₂O (0.01143 atm). Pressure control mechanism 74 may be a back pressure valve, a relief valve, backpressure regulator, or other similar device to regulate upstream (back) pressure. For example, pressure control mechanism 74 may be a model 9005B ACCU-PEEP™ back pressure valve manufactured by Vital Signs, Inc, Totowa, N.J., although other pressure control mechanisms may be used.

Control system 14b is adapted to be coupled via a counterlung 76 to hood 12. Counterlung 76 includes a first inlet port 78 and a second inlet port 80 coupled to first outlet port 48 and third outlet port 70, respectively, of control system 14b, and an outlet port 82 coupled via hose 42 and coupling mechanism 30 to inlet port 28 of hood 12. Counterlung 76 receives oxygen flow at its two inlet ports, and provides a single output oxygen flow at outlet port 82. Counterlung 76 may be a model 5063NL counterlung manufactured by Vital Signs, Inc, Totowa, N.J., although other counterlungs may be used.

Referring now to FIGS. 1 and 3, switch 60 may be used turn ON the flow of oxygen from oxygen supply 16. Filter 62 removes particulate or other unwanted matter in the flow of oxygen, which is regulated by flow control mechanism 64 to provide a desired flow rate via first outlet port 48 to counter lung 76. Counter lung 76 provides a flow of oxygen to hood 12 via hose 42. Oxygen begins filling chamber 40, and user 18 inhales oxygen from chamber 40, and exhales fluid (e.g., carbon dioxide) into chamber 40.

The exhaled fluid and residual oxygen returns via tube 44 and second inlet port 50 to pressure control mechanism 66, which regulates pressure inside chamber 40 to a desired microbaric pressure level. Any excess flow of return fluid is exhausted to CO₂ scrubber 72 and pressure control mechanism 74. CO₂ scrubber 76 substantially removes carbon dioxide from the return fluid, and provides oxygen to second inlet port 80 of counterlung 76. Thus, rather than being exhausted to atmosphere, oxygen in the return fluid is re-circulated to hood 12. Any excess flow of return fluid is exhausted to atmosphere by pressure control mechanism 74 via second outlet port 52 and tube 56.

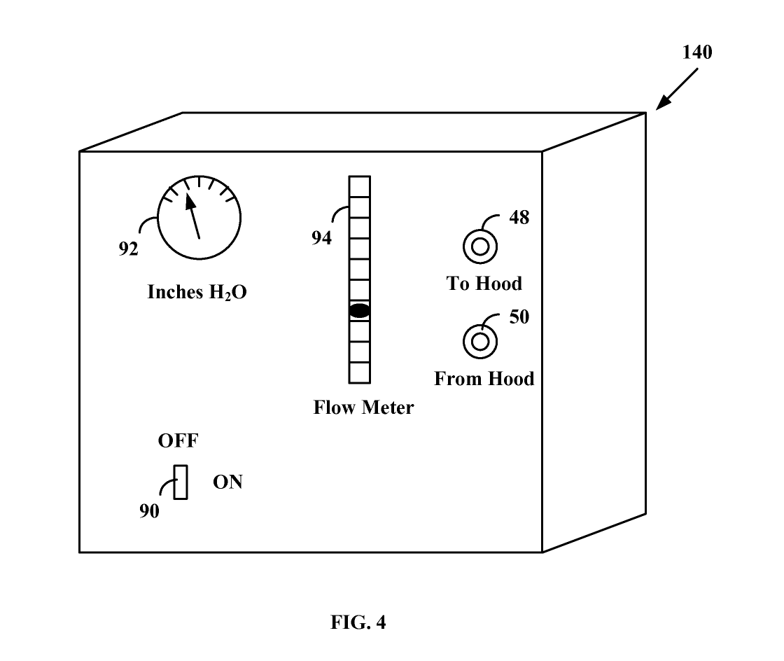

Referring now to FIG. 4, an example equipment housing 140 of control system 14 is described. Equipment housing 140 includes switch 90, pressure gauge 92, flow meter 94, outlet port 48 and inlet port 50. Although not shown in FIG. 4, equipment housing 140 also may include an inlet port 46 for coupling to oxygen supply 16 and an outlet port 52 for exhausting the system to atmosphere, as described above in connection with FIG. 1. Equipment housing 140 may be made from one or more of metal, plastic, wood, or other similar material.

Switch 90 may be any conventional switch that may be coupled to switch 60 (FIG. 2) for turning ON and OFF the flow of oxygen from oxygen supply 16. Pressure gauge 92 may be any conventional gauge for displaying pressure in chamber 40 of hood 12. For example, pressure gauge 92 may be a Dwyer Minihelic 2-5000-0 pressure gauge manufactured by Dwyer Instruments, Inc., Michigan City, Ind., although other pressure gauges may be used. Flow meter 94 may be any conventional meter for displaying flow of oxygen to hood 12. For example, flow meter 94 may be a GFM-1111 flow meter manufactured by Dwyer Instruments, Inc., Michigan City, Ind., although other flow meters may be used. Persons of ordinary skill in the art will understand that equipment housing 140 may include additional or fewer items than those shown in FIG. 4.

Referring again to FIG. 1, microbaric oxygen delivery system 10 may be used to provide microbaric oxygen delivery for treatment of acute and/or chronic neurological conditions, such as cerebral palsy, autism, traumatic brain injury, stroke, spinal cord injury, chronic fatigue syndrome, fibromyalgia, reflex sympathetic dystrophy, migraine and cluster headaches, multiple sclerosis, certain types of dementia, and other neurologic conditions.

For example, for microbaric oxygen treatment of autism, one or more tests may be used to determine a patient's motivation, speech, cognitive functiom, gross motor function, fine motor function, or other similar abilities, and/or spasticity or other similar conditions prior to commencing microbaric oxygen treatment. In this manner, baseline measurements and assessments of the patient's degree of neurologic impairment may be determined. After this baseline evaluation, microbaric oxygen delivery system 10 may be used to provide microbaric oxygen treatments to the patient for a particular duration, at a specified frequency, for a particular time period.

For example, the patient may be administered microbaric oxygen treatments lasting an hour per day, every day for 40 consecutive treatments. At that point, the same cognitive function, motor function, or other tests used to establish baseline measurements may be re-administered to assess the efficacy of the treatment. Based on the results, microbaric oxygen treatments may resume, or some treatment parameter (e.g., duration, frequency, or other parameter) may be modified. This treatment/assessment process may be repeated iteratively to fine-tune treatment parameters based on the patient's response to treatment.

Although an example has been described for microbaric oxygen treatment of autism, persons of ordinary skill in the art will understand that similar treatment protocols may be used to provide microbaric oxygen treatment of other neurological conditions, such as cerebral palsy, traumatic brain injury, stroke, spinal cord injury, chronic fatigue syndrome, fibromyalgia, reflex sympathetic dystrophy, migraine and cluster headaches, multiple sclerosis, certain types of dementia, and other neurologic conditions.

The foregoing merely illustrates the principles of this invention, and various modifications can be made by persons of ordinary skill in the art without departing from the scope and spirit of this invention.

1. A system for providing oxygen from an oxygen supply to a user, the system comprising:
a hood adapted to fit over the user's head, the hood comprising an inlet port, an outlet port, and a seal mechanism adapted to engage at least one of the user's head, neck and torso to restrict a flow of oxygen from the hood;
a flow control mechanism adapted to couple between the oxygen supply and the inlet port, and to control a flow of oxygen to the hood; and
a pressure control mechanism coupled to the outlet port, wherein the pressure control mechanism is adapted to regulate a pressure of the oxygen in the hood to between about 0.5 inches of water (“inH₂O”) (0.00127 atm) and about 4 inH₂O (0.01016 atm).

2. The system of claim 1, wherein the hood comprises one or more of glass, plastic, vinyl, Plexiglas and metal.

3. The system of claim 1, wherein the seal mechanism comprises one or more of a plastic, Teflon and rubber.

4. The system of claim 1, further comprising a first filter adapted to couple between the oxygen supply and the flow control mechanism, and to filter the oxygen supplied to the hood.

5. The system of claim 1, further comprising a second filter coupled between the outlet port and the pressure control mechanism, wherein the second filter is adapted to filter a fluid returned from the hood.

6. The system of claim 1, further comprising a switch adapted to couple between the oxygen supply and the flow control mechanism, and to control a flow of oxygen to the flow control mechanism.

7. The system of claim 1, further comprising a gauge adapted to display the pressure of oxygen in the hood.

8. The system of claim 1, further comprising a flow meter adapted to display information about the flow of oxygen to the hood.

9. The system of claim 1, further comprising an exhaust port coupled to the pressure control mechanism to exhaust a fluid returned from the hood to atmosphere.

10. A method for providing oxygen from an oxygen supply to a user, the method comprising:
providing a hood adapted to fit over the user's head, the hood comprising an inlet port, an outlet port, and a seal mechanism adapted to engage at least one of the user's head, neck and torso to restrict a flow of oxygen from the hood;
controlling a flow of oxygen to the inlet port of the hood; and
regulating a pressure of the oxygen in the hood from the outlet port to between about 0.5 inches of water (“inH₂O”) (0.00127 atm) and about 4 inH₂O (0.01016 atm).

11. The method of claim 10, wherein the hood comprises one or more of glass, plastic, vinyl, Plexiglas and metal.

12. The method of claim 10, wherein the seal mechanism comprises one or more of a plastic, Teflon and rubber.

13. The method of claim 10, further comprising filtering the oxygen supplied to the hood.

14. The method of claim 10, further comprising filtering a fluid returned from the hood.

15. The method of claim 10, further comprising providing a switch adapted to couple between the oxygen supply and the flow control mechanism to control a flow of oxygen to the flow control mechanism.

16. The method of claim 10, further comprising displaying the pressure of oxygen in the hood.

17. The method of claim 10, further comprising displaying information about the flow of oxygen to the hood.

18. The method of claim 10, further comprising exhausting a fluid returned from the hood to atmosphere.

19. A system for use with a counterlung for providing oxygen from an oxygen supply to a user, the counter lung having a first inlet port, a second inlet port, and an outlet port, the system comprising:
a hood adapted to fit over the user's head, the hood comprising an inlet port, an outlet port, and a seal mechanism adapted to engage at least one of the user's head, neck and torso to restrict a flow of oxygen from the hood;
a flow control mechanism adapted to couple between the oxygen supply and the first inlet port of the counter lung, and to control a flow of oxygen to the hood;
a first pressure control mechanism coupled to the outlet port of the hood, wherein the first pressure control mechanism is adapted to regulate a pressure of the oxygen in the hood to between about 0.5 inches of water (“inH₂O”) (0.00127 atm) and about 4 inH₂O (0.01016 atm); and
a carbon dioxide scrubber adapted to couple between the second inlet port of the counter lung and an outlet port of the first pressure control mechanism.

20. The system of claim 19, further comprising a second pressure control mechanism coupled to the outlet port of the first back pressure valve, wherein the second pressure control mechanism is adapted to regulate a pressure of oxygen in the system.
